(19) United States
(12) Patent Application Publication
(10) Pub. No.: US 2018/0296405 A1
(43) Pub. Date: Oct. 18, 2018

(54) WOUND PROTECTION DEVICE

(71) Applicant: Sherry May Raymond-Coblantz, Bend, OR (US)

(72) Inventor: Sherry May Raymond-Coblantz, Bend, OR (US)

(21) Appl. No.: 15/489,554

(22) Filed: Apr. 17, 2017

(51) Int. Cl. A61F 15/00; A61F 13/02

(52) U.S. Cl. CPC A61F 15/008; A61F 13/02

(57) ABSTRACT

A wound protection device can include a frame having a plurality of first members and a plurality of second members. The first members can be spaced relative to each other. The second members can be spaced relative to each other and can be coupled to the first members. The first members and the second members can define a plurality of a gaps through which a fluid can enter the frame. The frame can be configured to circumscribe a wound.

### FIELD

This disclosure relates generally to medical devices and more particularly to devices and methods for treating wounds or other skin conditions.

### BACKGROUND

A wound is a type of injury in which a person's skin is cut or broken. A person can get a wound in various ways such as by a medical condition, accident, or having a surgery, to name a few. Wounds of one kind or another affect virtually everyone at some point. Wounds can be painful, gruesome, and difficult to treat.

### SUMMARY

Disclosed herein are various embodiments of wound protection devices and related methods. The disclosed wound protection devices and methods can protect a wound while also allowing air circulation around the wound. Thus, the disclosed devices and methods can, for example, help the wound to heal quickly and properly.

In one representative embodiment, a wound protection device can include a frame having a plurality of first members and a plurality of second members. The first members can be spaced relative to each other. The second members can be spaced relative to each other and can be coupled to the first members. The first members and the second members can define a plurality of a gaps through which a fluid can enter the frame. The frame can be configured to circumscribe a wound.

In some embodiments, the first members comprise an annular shape, the second member comprise an arcuate shape, and the frame comprises a dome shape.

In some embodiments, the frame comprises adhesive configured for attaching the wound protection device to a user's skin. In some embodiments, one or more of the first members comprises the adhesive.

In some embodiments, the wound protection device further comprises a cover that extends over the frame such that the fluid cannot enter the frame. In some embodiments, the cover is releasably coupled to the frame.

In some embodiments, the frame comprises a groove configured to releasably receive an edge portion of the cover. In some embodiments, one of the first members of the frame comprises the groove.

In some embodiments, the frame comprises one or more openings through which medication can be applied. In some embodiments, the openings comprise a conical shape taken in a plane perpendicular to a longitudinal axis of the opening.

In some embodiments, the first members of the frame are configured to direct the fluid toward the wound. In some embodiments, each of the first members of the frame has a conical shape. In some embodiments, each of the first members extends from a respective second member at an angle, and the angle between the first member and a vertical plane extending through the respective second member is between 70 and 80 degrees.

In some embodiments, the frame comprises an hourglass shape and is configured to be attached a pelvic region of a user.

In some embodiments, the fluid comprises air.

In another representative embodiment, a wound protection device can comprise a frame and a cover. The frame can have a plurality of openings and can be configured to protect a wound and to allow air to flow to the wound through the openings. The cover can be configured to be releasably coupled to the frame and to seal the openings of the frame.

In some embodiments, the frame comprises a groove configured to receive an edge portion of the cover.

In some embodiments, the frame comprises a plurality of first members and a plurality of second members coupled to the first members, wherein one or more of the first members extend from respective second members at an angle, and the angle between the one or more first members and a vertical plane extending through the respective second members can be between 0 and 90 degrees, such as, for example 70 to 80 degrees or, more particularly, 75 or 76 degrees.

In another representative embodiment, a wound protection device can comprise a plurality of first members and a plurality of second members. The first members can be spaced relative to each other, and the second members can be spaced relative to each other and can be coupled to the first members. The first members and the second members can define a plurality of a gaps through which a fluid can flow. The wound protection device can be configured to be attached a pelvic region of a user.

In some embodiments, one or more of the first members comprise an hourglass shape, and the second members comprise an arcuate shape.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, which proceeds with reference to the accompanying figures.

### BRIEF DESCRIPTION OF THE DRAWINGS

FIG. 1 shows an exemplary embodiment of a wound protection device.

FIG. 2 is a top plan view of the wound protection device of FIG. 1.

FIG. 3 is a bottom plan view of the wound protection device of FIG. 1.

FIG. 4 is a cross-sectional view of the wound protection device of FIG. 1, taken along the line 4-4 as illustrated in FIG. 1.

FIG. 5 is a perspective view of the wound protection device of FIG. 1, showing the device attached to an arm of a user.

FIG. 6 shows an exemplary embodiment of a cover for the wound protection device of FIG. 1, which is attached to the arm of the user.

FIG. 7 shows another exemplary embodiment of a wound protection device.

### DETAILED DESCRIPTION

For purposes of this description, certain aspects, advantages, and novel features of the embodiments of this disclosure are described herein. The described methods, systems, and apparatus should not be construed as limiting in any way. Features, characteristics, and/or groups described in conjunction with a particular aspect, embodiment or example are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The explanations of terms and abbreviations herein are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

As used herein, the term “and/or” used between the last two of a list of elements means any one of, or any combination of, the listed elements. For example, the phrase “A, B, and/or C” means “A,” “B,” “C,” “A and B,” “A and C,” “B and C,” or “A, B, and C.”

As used herein, the terms “attached” and “coupled” generally mean physically connected or linked, which includes items that are directly attached/coupled and items that are attached/coupled with intermediate elements between the attached/coupled items, unless specifically stated to the contrary.

As used herein, the term “air” generally means fluid comprising nitrogen, oxygen, and/or other fluids typically found in the ambient environment.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the detailed description, claims, abstract, and drawings.

As mentioned above, a person can get a wound in various ways such as by a medical condition (e.g., dermatitis), accident (e.g., falling), or having a surgery, to name a few, and wounds can be painful, gruesome, and difficult to treat. Typical wound treatment includes covering the wound with a bandage. Bandages have several disadvantages. For example, bandages can prevent or severely restrict airflow to a wound, which can prevent or impede healing. This is because the body's fibroblasts, which are cells that play a key role in tissue regeneration, work better with access to air. Bandages can also apply pressure on the wound, thus causing pain and irritation. Bandages can also cause pain and irritation through movement and/or rubbing on clothing.

To avoid the disadvantages of bandages, many people forego using them. This can, however, cause other problems. For example, leaving the wound completely uncovered can cause the wound to become contaminated, which can lead to infection. The wound can also catch or snag on objects such as clothing, which can cause the wound to re-open. In addition, many people tend to “dig” or “pick” at their wounds when the wound is left uncovered. This can re-open the wound and lead to infection.

Due to the disadvantages of typical bandages and of leaving the wound completely uncovered, improved wound protection devices are needed.

Disclosed herein are various embodiments of wound protection devices and related methods. The disclosed wound protection devices and methods can protect a wound while also allowing air circulation around the wound. The disclosed devices and methods can also allow medication to be applied to a protected wound. Thus, the devices and methods can, for example, help the wound to heal quickly and properly.

For example, in some embodiments, the wound protection device can comprise a frame configured to protect a wound. The frame can comprise a plurality of openings which can allow fluid (e.g., oxygen, air, and/or medication) to enter into the frame.

In some embodiments, the wound protection device can comprise a cover releasably coupled to the frame. The cover can be configured to seal the openings in the frame. Thus, the cover can be used, for example, to help keep a wound moist. The cover can also be used, for example, to prevent substance (water, dirt, and/or other substances) from entering the frame and contaminating the wound (e.g., during showering).

In addition to wounds, the disclosed devices and methods can be used to treat other medical conditions in which an area of the body needs protection and airflow. For example, in some embodiments, a device can be configured to treat other forms of damaged skin such as psoriasis, cantankerous hook, and/or other types of skin lesions.

In certain embodiments, a device can be configured to treat “urethral syndrome,” also known as “symptomatic abacteriuria,” and “interstitial cystitis,” also called “bladder pain syndrome.” Both of these conditions can cause bladder pressure, bladder pain, and sometimes pelvic pain.

For example, in some embodiments, the wound protection device can comprise a frame configured to reduce or prevent pressure on the urethra. The frame can comprise a plurality of openings which can allow air to enter into the frame.

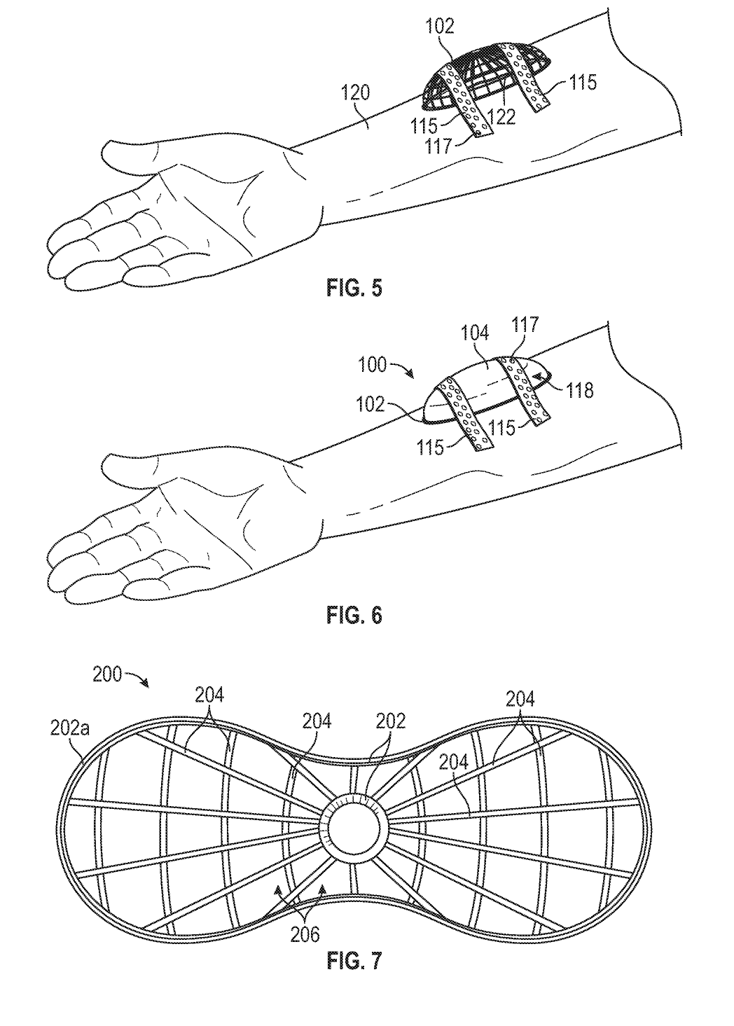

FIGS. 1-6 show an exemplary embodiment of a wound protection device 100 (FIG. 6) and its components. Referring to FIGS. 5-6, the wound protection device 100 can comprise a frame 102 (FIGS. 1-6) and an optional cover 104 (FIG. 6). As best shown in FIG. 5, the frame 102 can be configured to protect a wound, while still allowing airflow to the wound. The cover 104 can be releasably coupled to the frame 102 and can be configured to temporarily cover the frame 102 and thus seal the wound, as shown in FIG. 6.

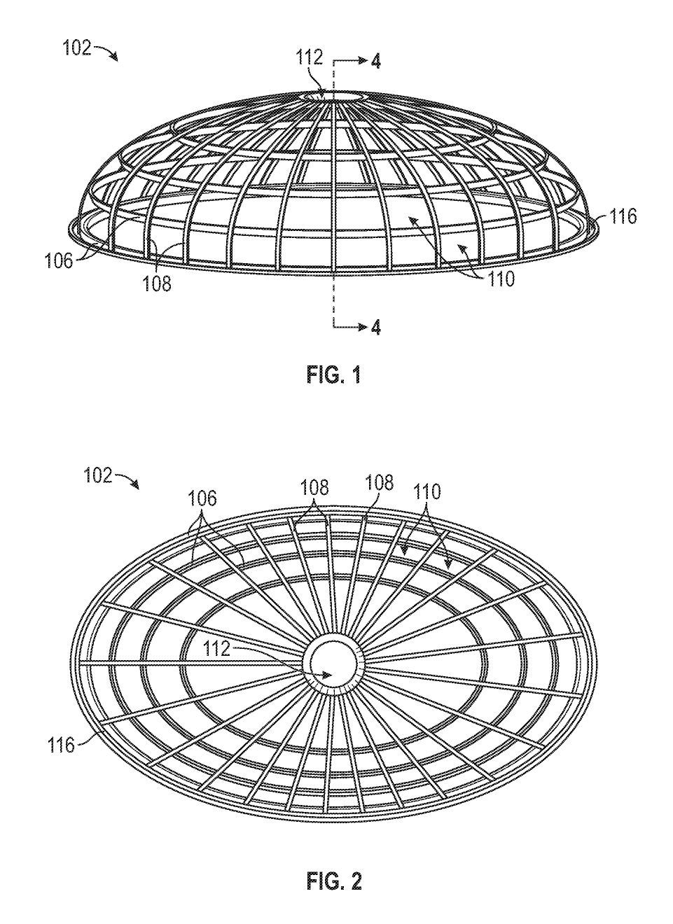

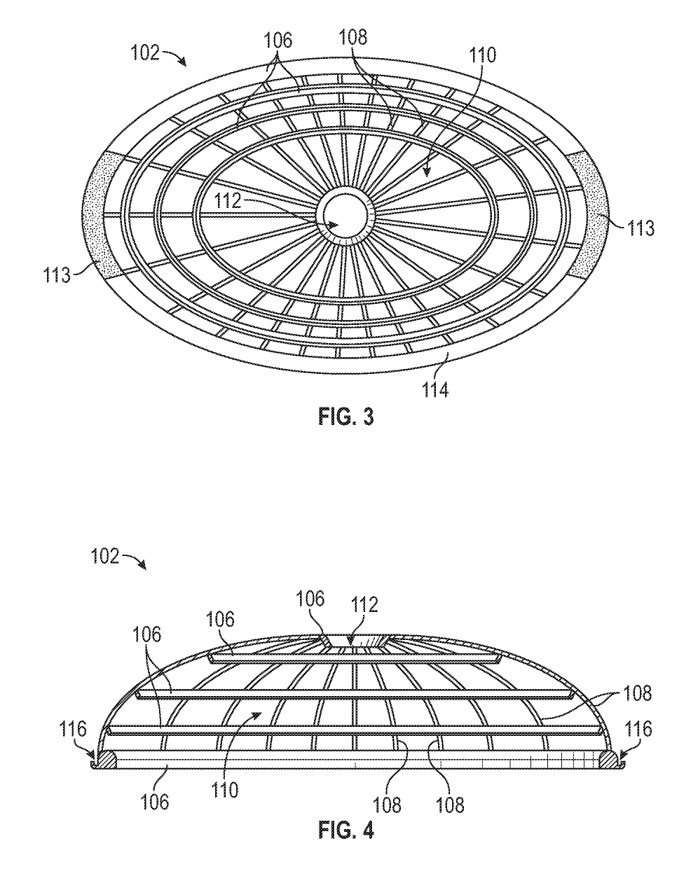

Referring to FIGS. 2-3, the frame 102 of the wound protection device 100 can comprise a plurality of first members 106 (e.g., 5 in the illustrated embodiment) and a plurality of second members 108 (e.g., 25 in the illustrated embodiment). Each of the first members 106 can extend annularly, and each of the second members 108 can extend laterally over and can be coupled to the first members 106. As best shown in FIG. 4, the first members 106 can be laterally (e.g., vertically in the orientation shown in FIG. 4) spaced relative to each other. As best shown in FIG. 2, the second members 108 can be circumferentially spaced relative to each other around the first members 106. In this manner, the first and the second members 106, 108 of the frame 102 form a lattice-type structure having a plurality of gaps or open-cells 110 defined by the first and second members 106, 108.

The frame 102 can be configured and sized such that the gaps 110 allow fluid (e.g., air) to flow through the frame 102 and prevent other objects (e.g., the user's finger) from entering therethrough. In some embodiments, for example, the frame 102 can be configured with fewer or more first and/or second members 106, 108 to provide a desired size for the gaps 110. For example, providing few first and/or second members 106, 108 can increase the size of the gaps 110, and providing more first and/or second members 106, 108 can decrease the size of the gaps 110. In certain embodiments, a height, width, thicknesses, and/or other dimension of the first and/or second members 106, 108 can configured to size the gaps 110 as desired. For example, first and/or second members 106, 108 with relatively larger dimensions provide relatively smaller gaps 110, and first and/or second members 106, 108 with relatively smaller dimensions provide relatively larger gaps 110.

In certain embodiments, one or more of the first members 106 can be configured to overlap with adjacent first members 106. In this manner, the gaps 110 would not be visible when viewing the frame 102 from the side (e.g., from the perspective shown in FIG. 4). Nevertheless, fluid can flow through the gaps 110 to the wound.

The frame 102 can be configured to have smooth profile such that there are not any shape edges exposed so as to prevent the frame from catching or snagging on an object (e.g., a user's clothes). For example, as shown in the illustrated embodiment, the frame 102 can be configured in a dome shape. In such embodiments, the second members 108 can extend arcuately between the first 106, as best shown in FIGS. 1 and 4. In other embodiments, the frame 102 can be configured in various other shapes such as pyramidal, trapezoidal prism, rectangular prism, or other suitable shape.

The frame 102 can also comprise one or more openings 112 (e.g., one in the illustrated embodiment). The opening 112 can be defined by one of the first members 106 (e.g., the uppermost first member 106), as best shown in FIG. 4. The opening 112 can be configured and sized such that ointment can be applied to a wound therethrough. For example, the opening 112 can be configured and sized to receive a medicine dropper. In some embodiments, the opening 112 can be configured like a funnel (e.g., having a conical cross-sectional profile taken in a plane perpendicular to a longitudinal axis of the opening 112), as best shown in FIG. 4.

As also shown in FIG. 4, in some embodiments, the first members 106 can comprise a conical shape such that the first members 106 extend radially inwardly from the second members 108. In other words, each first member 106 has a first (e.g., the upper) edge portion that has a diameter that is larger than a diameter of a second (e.g., the lower) edge portion. In this manner, the first members 106 are configured like a funnel and/or fins (e.g., similar to the opening 112), which can help to direct airflow into the frame 102 and toward a wound.

The first members 106 can extend from the second members 108 at various angles. For example, in some embodiments, the angle between each first member 106 and a vertical plane extending through the respective second member 108 can be between 0 and 90 degrees, for example, between 10 and 80 degrees, or specifically 75 or 76 degrees.

In some embodiments, one of the first members 106 (e.g., the lowermost first member 106) can be configured to press and/or seal against a user's skin. In certain embodiments, the first member 106 that contacts the user's skin can comprise adhesive 113, e.g., on a skin-facing surface 114 (FIG. 3). The adhesive 113 can be configured for releasably attaching the frame 102 to the user's skin. The adhesive 113 can, for example, be a biocompatible, pressure-sensitive adhesive covered by release paper. The release paper can protect the adhesive 113 until a user removes the release paper and presses the adhesive against their skin, thus attaching the frame 102 to their skin.

In lieu of or in addition to the adhesive, the frame 102 can also be attached to a user's skin with medical tape, a strap or band, and/or other means for attaching. In some embodiments, tape 115 comprising perforations 117 can be used to attach the frame 102 and/or the cover to the user's skin, as shown in FIGS. 5 and 6. The perforations 117 can, for example, allow airflow through the tape 115.

In certain embodiments, the tape 115 can be relatively narrow (e.g., ⅛-½ inch). In such embodiments, the perforations 117 can be comprise an oblong shape (e.g., oval, rectangular). The perforations 117 can be oriented such that a major axis of the oblong shape is aligned with a length direction of the tape 115 and a minor axis of the oblong shape is aligned with a width direction of the tape 115. Configuring the perforations 117 in this manner can, for example, allow the perforations 117 to be relatively large for a relatively narrow width of tape 115. The large perforations 117 and/or narrow width of the tape 115 can, for example, improve breathability of the tape 115.

In some embodiments, the tape 115 can comprise various other widths (e.g., ½-2 inches) and can be configured for various applications. In some embodiments, the perforations 117 can comprise various other shapes, such as circular or square to name a few.

In some embodiments, the frame 102 can comprise a mating feature configured such that the cover 104 can be releasable coupled to the frame 102. For example, as shown in FIG. 4, one of the first members 106 (e.g., the lowermost first member 106) can comprise a groove 116. The groove 116 can be configured to releasably receive an edge portion 118 of the cover 104 in a snap-fit type connection, as best shown in FIG. 6.

The frame 102 can be formed in various ways. For example, in certain embodiments, the first and second members 106, 108 of the frame 102 can be integrally formed as a single, unitary component (e.g., by co-molding). In other embodiments, the first and second members 106, 108 can be formed as separately pieces that are coupled together with adhesive, fasteners, and/or other means for coupling.

For example, the first members 106 can be coupled to inwardly facing surfaces of the second members 108, as shown in the illustrated embodiment. In other embodiments, the first members 106 can be coupled to outwardly facing surfaces of the second members 108. In yet other embodiments, the first members 106 can be coupled to the second members 108 between the inwardly facing surfaces and the outwardly facing surfaces of the second members 108.

In some embodiments, the frame 102 can configured to be relatively flexible so as to conform to contours of the user's body. For example, frame 102 can flex to conform to a user's arm, leg, back, etc.

In some embodiments, the frame 102 can also be configured to be relatively rigid such that the frame will not compress inwardly against a wound under typical forces that are applied to the frame. For example, the frame 102 can be configured to withstand forces applied to the frame 102 by the user's clothing, when the user bumps or rests their arm on an object, and/or when the user falls on frame 102.

As shown in FIG. 5, for example, the frame 102 of the wound protection device 100 can be attached to a user's arm 120 at a location circumscribing a wound 122. The frame 102 can be attached by the adhesive on the frame 102 and/or by medical tape 124 extending from and/or over the frame 102. In this manner, the frame 102 can be used to protect a wound by preventing the wound from contacting the user's clothing and/or other objects, while still allowing airflow to the wound.

As shown in FIG. 6, the cover 104 can be releasable coupled to the frame 102. The cover 104 can be configured to extend over the frame 102 to seal the gaps 110 in the frame 102. Accordingly, the cover 104 can be used, for example, to seal the wound while a user is in the shower and/or in an environment in which the wound could be contaminated (e.g., by dirt, debris, etc.). The cover 104 can also be used, for example, to keep a wound moist by retaining moisture within the cover 104 (e.g., after applying certain ointments that require the wound to be covered after application of the ointment).

The wound protection device 100 can be formed from various biocompatible materials. For example, in some embodiments, the frame 102 and/or the cover 106 of the wound protection device 100 can be formed from polymers, metals, composites, and/or other suitable biocompatible materials.

FIG. 7 shows exemplary embodiment of a wound protection device 200. The wound protection device can comprise a plurality of first members 202 (e.g., two in the illustrated embodiment and a plurality of second member 204 (e.g., 22 in the illustrated embodiment). The first members 202 can extend annularly and can be spaced relative to each other. The second members 204 can be coupled to the first members 202 and can be spaced relative to each other. In this manner, the first and the second members 202, 204 of the wound protection device 200 form a lattice-type structure having a plurality of gaps or open-cells 206 defined by the first and second members 202, 204.

In some embodiments, the wound protection device 200 can be configured for to protect a pelvic or groin region of the body. For example, in certain embodiments, the one or more of the first members 202 (e.g., the outermost first member 202a) a can comprise an “hourglass” or “figure-8” shape.

In this manner, the wound protection device 200 can be used, for example, to treat urethral syndrome and/or interstitial cystitis by protecting the pelvic region from pressure and/or contact clothing, seats, etc., while also allowing airflow to the area.

The features described herein with regard to any example can be combined with other features described in any one or more of the other examples, unless otherwise stated. For example, components, materials and/or properties of the wound protection device 100 can be combined with the components, materials, and/or properties of the wound protection device 200.

In view of the many possible embodiments to which the principles of the disclosure may be applied, it should be recognized that the illustrated embodiments are only preferred examples and should not be taken as limiting the scope of the claims. Rather, the scope of the claimed subject matter is defined by the following claims and their equivalents.

1. A wound protection device, comprising:
a frame having a plurality of first members and a plurality of second members,
wherein the first members are spaced relative to each other,
wherein the second members are spaced relative to each other and are coupled to the first members,
wherein the first members and the second members define a plurality of a gaps through which a fluid can enter the frame, and
wherein the frame is configured to circumscribe a wound.

2. The device of claim 1, wherein the first members comprise an annular shape, the second member comprise an arcuate shape, and the frame comprises a dome shape.

3. The device of claim 1, wherein the frame comprises adhesive configured for attaching the wound protection device to a user's skin.

4. The device of claim 4, wherein one or more of the first members comprises the adhesive.

5. The device of claim 1, further comprising a cover that extends over the frame such that the fluid cannot enter the frame.

6. The device of claim 5, wherein the cover is releasably coupled to the frame.

7. The device of claim 6, wherein the frame comprises a groove configured to releasably receive an edge portion of the cover.

8. The device of claim 7, wherein one of the first members of the frame comprises the groove.

9. The device of claim 1, wherein the frame comprises one or more openings through which medication can be applied.

10. The device of claim 9, wherein the openings comprise a conical shape taken in a plane perpendicular to a longitudinal axis of the opening.

11. The device of claim 1, wherein the first members of the frame are configured to direct the fluid toward the wound.

12. The device of claim 11, wherein each of the first members of the frame has a conical shape.

13. The device of claim 12, wherein each of the first members extends from a respective second member at an angle, and the angle between the first member and a vertical plane extending through the respective second member is between 70 and 80 degrees.

14. The device of claim 1, wherein the frame comprises an hourglass shape and is configured to be attached a pelvic region of a user.

15. The device of claim 1, wherein the fluid comprises air.

16. A wound protection device, comprising:
a frame having a plurality of openings, wherein the frame is configured to protect a wound and to allow air to flow to the wound through the openings; and
a cover releasably coupled to the frame, wherein the cover is configured to seal the openings of the frame.

17. The device of claim 16, wherein the frame comprises a groove configured to receive an edge portion of the cover.

18. The device of claim 16, wherein the frame comprises a plurality of first members and a plurality of second members coupled to the first members, wherein one or more of the first members extend from respective second members at an angle, and the angle between the one or more first members and a vertical plane extending through the respective second members is between 0 and 90 degrees.

19. A wound protection device, comprising:
a plurality of first members spaced relative to each other; and
a plurality of second members, wherein the second members are spaced relative to each other and are coupled to the first members,
wherein the first members and the second members define a plurality of a gaps through which a fluid can flow, and
wherein the wound protection device is configured to be attached a pelvic region of a user.

20. The device of claim 19, wherein one or more of the first members comprise an hourglass shape, and wherein one or more of the second members comprise an arcuate shape.
